(19) United States
(12) Patent Application Publication
(10) Pub. No.: US 2015/0209004 A1
(43) Pub. Date: Jul. 30, 2015

(54) ULTRASONIC MEASUREMENT APPARATUS AND ULTRASONIC MEASUREMENT METHOD

(71) Applicant: SEIKO EPSON CORPORATION, Tokyo (JP)

(72) Inventor: Natsumi TAMADA, Shiojiri-shi (JP)

(21) Appl. No.: 14/595,749

(22) Filed: Jan. 13, 2015

(30) Foreign Application Priority Data
Jan. 30, 2014 (JP) 2014-015167

(51) Int. Cl. A61B 8/08; A61B 8/14; A61B 8/00

(52) U.S. Cl. CPC A61B 8/0891; A61B 8/5223; A61B 8/54; A61B 8/461; A61B 8/467; A61B 8/14

(57) ABSTRACT

Scanning lines immediately above the blood vessel are detected using received signals of reflected waves obtained when ultrasonic waves transmitted to the blood vessel are reflected from the blood vessel, and candidates at depth positions that seem to be front and rear walls of the blood vessel are detected based on the received signals of the scanning lines. Then, vascular front and rear walls pairs of front and rear walls are narrowed down from the candidates, and the narrowed-down vascular front and rear walls pair is regarded as one blood vessel and artery/vein identification is performed for each blood vessel. Measurement of vascular function information is performed for the blood vessel determined to be an artery.

### BACKGROUND

1. Technical Field

The present invention relates to an ultrasonic measurement apparatus that performs measurement using an ultrasonic wave.

2. Related Art

As an example of measuring biological information with an ultrasonic measurement apparatus, the evaluation of a vascular function or the determination of a vascular disease is performed.

For example, the intima media thickness (IMT) of the carotid artery, which is an indicator of arteriosclerosis, is measured. In the measurement relevant to the IMT or the like, it is necessary to locate the carotid artery and appropriately determine the measurement point. Typically, the operator places an ultrasonic probe on the neck, locates the carotid artery to be measured while watching a B-mode image displayed on the monitor, and manually sets the found carotid artery as a measurement point.

Since skill is required in order to execute such a series of measurement operations quickly and locate the carotid artery appropriately in the related art, a function to assist the measurement operation has been devised in recent years. For example, JP-A-2008-173177 discloses a method of detecting the vessel wall automatically using the strength of a reflected wave signal from the body tissue, which is obtained by processing the amplitude information of the received reflected wave, and the moving speed of the body tissue, which is obtained by processing the phase information of the received reflected wave. Specifically, a boundary between the vessel wall and the blood flow region is detected based on the first finding that the strength of the reflected wave signal in the blood flow region in the blood vessel is very small compared with the strength of the reflected wave signal in the vessel wall and the second finding that the moving speed calculated from the phase information of the reflected wave signal is high in the blood flow region and low in the vessel wall.

However, in the detection method disclosed in JP-A-2008-173177, a blood vessel can be detected, but it is not possible to determine whether the blood vessel is an artery or a vein.

In general, the artery exhibits pulsation, but the vein does not exhibit pulsation. For this reason, the operator tends to think simply that the artery and the vein can be identified by the presence or absence of pulsation. However, in blood vessels relatively close to the heart, such as the internal jugular vein, even veins may exhibit pulsation due to the pressure of the right atrium being transmitted thereto. Therefore, it is difficult to perform correct identification from only the presence or absence of pulsation.

### SUMMARY

An advantage of some aspects of the invention is to realize a technique for identifying an artery and a vein.

A first aspect of the invention is directed to an ultrasonic measurement apparatus including: a transmission and reception control unit that controls transmission of ultrasonic waves to a blood vessel and reception of reflected waves; a front and rear walls detection unit that detects front and rear walls of the blood vessel using received signals of the reflected waves; and a type determination unit that determines a type of the blood vessel using a temporal change in a distance between the front and rear walls.

According to the first aspect of the invention, it is possible to identify arteries and veins.

A second aspect of the invention is directed to the ultrasonic measurement apparatus according to the first aspect of the invention, wherein the type determination unit determines a type of the blood vessel using a temporal change in the distance in a direction of increase and a temporal change in the distance in a direction of decrease.

A third aspect of the invention is directed to the ultrasonic measurement apparatus according to the second aspect of the invention, wherein the type determination unit determines a type of the blood vessel using a ratio between an extreme value of the temporal change in the direction of increase and an extreme value of the temporal change in the direction of decrease.

A fourth aspect of the invention is directed to the ultrasonic measurement apparatus according to the third aspect of the invention, wherein the type determination unit determines that the blood vessel is an artery using at least a value that the ratio can have when the blood vessel is an artery.

According to the second to fourth aspects of the invention, the determination is performed based on the temporal change in the entire blood vessel diameter. Therefore, even under the specific conditions in which one of the positions of the front and rear walls hardly moves, for example, depending on the state of the tissues around the blood vessel, it is possible to realize correct determination.

A fifth aspect of the invention is directed to the ultrasonic measurement apparatus according to any one of the first to fourth aspects of the invention, wherein the front and rear walls detection unit detects front wall candidates and rear wall candidates of the blood vessel using the received signals, and selects a pair satisfying predetermined conditions, among pairs of the front wall candidates and the rear wall candidates, as the front and rear walls of the blood vessel.

According to the fifth aspect of the invention, even in a place where a plurality of blood vessels are adjacent to each other, it is possible to identify each of the blood vessels and determine the type of each blood vessel.

A sixth aspect of the invention is directed to the ultrasonic measurement apparatus according to the fifth aspect of the invention, wherein the front and rear walls detection unit selects the front and rear walls of the blood vessel based on the predetermined conditions including at least a condition that a signal between each of the front wall candidates and each of the rear wall candidates, among the received signals, satisfies predetermined intravascular equivalent conditions.

According to the sixth aspect of the invention, it is possible to exclude the tissues in the body having similar ultrasonic wave reflection characteristics to the vessel wall and to appropriately select the front and rear walls of the blood vessel.

A seventh aspect of the invention is directed to the ultrasonic measurement apparatus according to any one of the first to sixth aspects of the invention, which further includes a vascular function measuring unit that performs predetermined vascular function measurement by continuing position measurement with the front and rear walls of the blood vessel as tracking targets when the blood vessel is determined to be an artery by the type determination unit.

According to the seventh aspect of the invention, it is possible to realize a series of processes for automatically locating the artery and performing vascular function measurement for the artery.

An eighth aspect of the invention is directed to an ultrasonic measurement method including: controlling transmission of ultrasonic waves to a blood vessel and reception of reflected waves; detecting front and rear walls of the blood vessel using received signals of the reflected waves; and determining a type of the blood vessel using a temporal change in a distance between the front and rear walls.

According to the eighth aspect of the invention, it is possible to achieve the same effects as in the first aspect of the invention.

### BRIEF DESCRIPTION OF THE DRAWINGS

The invention will be described with reference to the accompanying drawings, wherein like numbers reference like elements.

FIG. 1 is a diagram showing an example of the system configuration of a biological information measuring apparatus.

FIG. 2 is a flowchart showing the flow of the main process performed by an ultrasonic measurement apparatus.

FIG. 3 is a diagram schematically showing a state where an ultrasonic probe is in contact with the body surface of a subject in order to perform ultrasonic measurement, and is a diagram showing the cross-section of a blood vessel in a short-axis direction.

FIGS. 4A to 4C are diagrams showing an example of the received signal of the reflected wave at the position of an ultrasonic transducer located immediately above the blood vessel.

FIGS. 5A and 5B are diagrams for explaining the statistical processing on a change in the signal strength between two consecutive frames.

FIGS. 6A to 6C are diagrams for explaining the principle of the detection of a vessel wall depth position candidate.

FIGS. 7A and 7B are graphs showing an example of a change in the blood vessel diameter for approximately one beat of the cardiac cycle, where FIG. 7A is a graph of the arterial blood vessel diameter and FIG. 7B is a graph of the venous blood vessel diameter.

FIG. 8A is a diagram showing a displacement rate waveform of the artery wall for approximately three beats of the cardiac cycle, FIG. 8B is a diagram showing a diameter change rate waveform of the artery diameter for approximately three beats of the cardiac cycle, and FIG. 8C is a diagram showing the ratio between the absolute values of extreme values (maximum and minimum values), that is, the peak ratio (maximum value/minimum value) in the diameter change rate waveform.

FIG. 9A is a diagram showing a displacement rate waveform of the vein wall for approximately three beats of the cardiac cycle, FIG. 9B is a diagram showing a diameter change rate waveform of the vein diameter for approximately three beats of the cardiac cycle, and FIG. 9C is a diagram showing the ratio between the absolute values of extreme values (maximum and minimum values), that is, the peak ratio (maximum value/minimum value) in the diameter change rate waveform.

FIG. 10 is a block diagram showing an example of the functional configuration of the ultrasonic measurement apparatus.

FIG. 11 is a diagram showing an example of a program or data stored in a storage unit.

FIG. 12 is a diagram showing an example of the data configuration of vascular front and rear walls pair data.

FIG. 13 is a flowchart for explaining the flow of the process of detecting the scanning lines immediately above the blood vessel.

FIG. 14 is a flowchart for explaining the flow of the process of detecting the vessel wall depth position candidate.

FIG. 15 is a flowchart for explaining the flow of the process of narrowing down the vascular front and rear walls pairs.

FIG. 16 is a flowchart for explaining the flow of the artery determination process.

### DESCRIPTION OF EXEMPLARY EMBODIMENTS

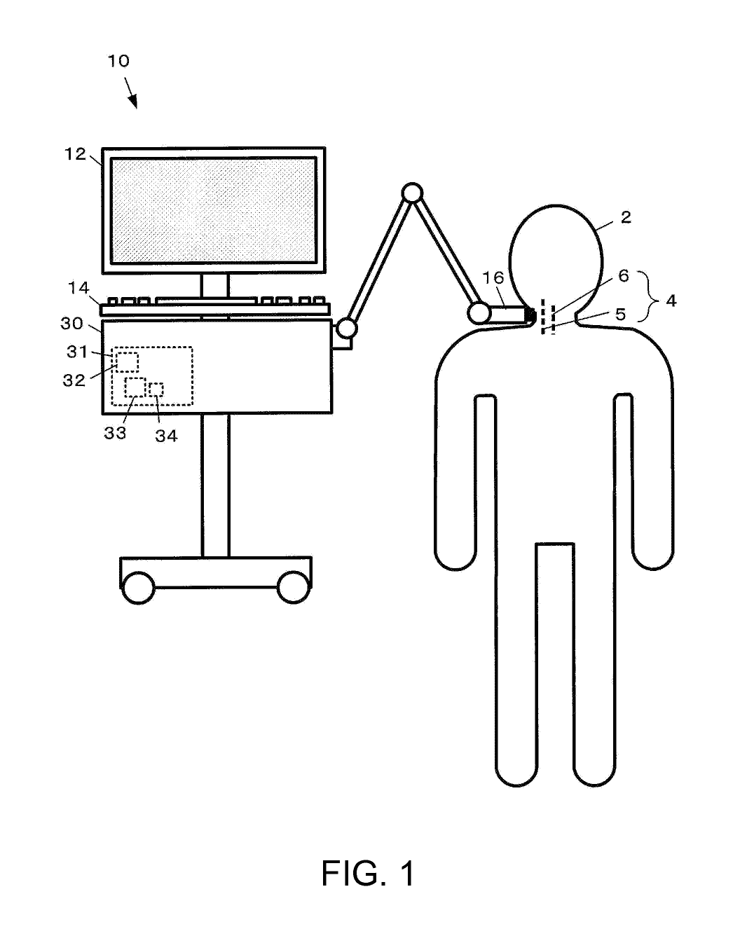

FIG. 1 is a diagram showing an example of the system configuration of an ultrasonic measurement apparatus 10 according to the present embodiment. The ultrasonic measurement apparatus 10 is an apparatus that measures biological information of a subject 2 by measuring the reflected waves of ultrasonic waves. In the present embodiment, an artery 5 and a vein 6 of blood vessels 4 are automatically identified, and vascular function information, such as the intima media thickness (IMT) of the artery 5, is measured as a piece of biological information.

The ultrasonic measurement apparatus 10 includes a touch panel 12 serving as a unit that displays a measurement result or operation information as an image and as an operation input unit, a keyboard 14 used for operation input, an ultrasonic probe 16, and a processor 30. A control board 31 is mounted in the processor 30, and is connected to each unit of the apparatus, such as the touch panel 12, the keyboard 14, and the ultrasonic probe 16, so that signal transmission and reception therebetween are possible.

Not only various integrated circuits, such as a central processing unit (CPU) 32 and an application specific integrated circuit (ASIC), but also a storage medium 33, such as an IC memory or a hard disk, and a communication IC 34 for realizing data communication with an external device are mounted on the control board 31. The processor 30 realizes various functions according to the present embodiment, such as identification of the arteries and veins, measurement of vascular function information for the identified artery 5, and image display control of the measurement result, including ultrasonic measurement by executing a measurement program stored in the storage medium 33 with the CPU 32 or the like.

Specifically, by the control of the processor 30, the ultrasonic measurement apparatus 10 transmits and emits an ultrasonic beam from the ultrasonic probe 16 to the subject and receives the reflected wave. Then, by performing amplification and signal processing on a received signal of the reflected wave, it is possible to generate reflected wave data, such as a temporal change or position information of a structure in the living body of the subject 2. Images in respective modes of so-called A mode, B mode, M mode, and color Doppler are included in the reflected wave data. Measurement using an ultrasonic wave is repeatedly performed at predetermined periods. The measurement unit is referred to as a “frame”.

By setting a region of interest (tracking point) in the reflected wave data as a reference, the ultrasonic measurement apparatus 10 can perform so-called “tracking” that is tracking each region of interest between different frames and calculating the displacement.

First, the outline of the process leading up to the measurement of vascular function information will be described.

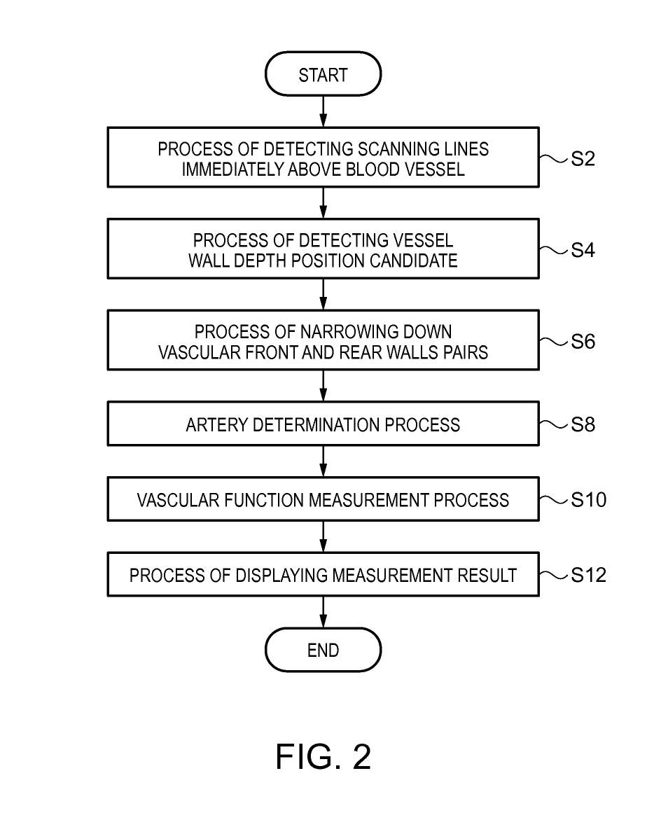

FIG. 2 is a flowchart showing the flow of the main process performed by the ultrasonic measurement apparatus 10. It is assumed that the ultrasonic probe 16 is directed toward the carotid artery by the operator. The ultrasonic measurement apparatus 10 detects an ultrasonic transducer (can also be a scanning line rather than the transducer) located immediately above the blood vessel regardless of the distinction of arteries and veins (step S2). This is referred to as a “scanning line immediately above the blood vessel”. In addition, “immediately above” referred to herein, needless to say, includes a position directly above the blood vessel center literally, but also has the meaning allowing a slight shift in a radial direction from the position immediately above in a range that is sufficient to measure the vascular function information of interest. “Immediately above” or “directly above” is not necessarily the meaning of an upward direction (opposite direction to gravity), but is the meaning in the operation of the operator who handles the ultrasonic probe 16 to place the ultrasonic probe 16 “immediately above” or “directly above” the blood vessel on the body surface.

Then, a candidate at a depth position that seems to be a vessel wall is detected from the reflected wave data in the scanning lines immediately above the blood vessel (step S4). Although a part regarded as the front wall (vessel wall facing the skin side) of the blood vessel or the rear wall (vessel wall located opposite the front wall) of the blood vessel is detected in this stage, a body part other than the blood vessels may be included in depth position candidates since the part has not yet been determined as a blood vessel. Therefore, the ultrasonic measurement apparatus 10 narrows down the pairs of front and rear walls of the blood vessels from the detected depth position candidates (step S6). The narrowed-down pair of depth position candidates are called a “vascular front and rear walls pair”.

Then, the ultrasonic measurement apparatus 10 performs artery determination for each narrowed-down vascular front and rear walls pair, thereby identifying whether the vascular front and rear walls pair corresponds to an artery or corresponds to a vein (step S8). For the vascular front and rear walls pair determined to be the artery 5, the ultrasonic measurement apparatus 10 performs vascular function measurement (step S10) . Then, the measurement result is displayed on the touch panel 12 (step S12). The content of the vascular function measurement may be other content without being limited to the IMT, and a known technique can be appropriately used.

Description of Principle

Next, each step will be described in detail.

First, a step of detecting the scanning lines immediately above the blood vessel will be described. The detection of the scanning lines immediately above the blood vessel is based on the movement of body tissues. That is, a blood vessel position is determined based on the finding that blood vessels move largely periodically with the beating of the heart but the movement of other body tissues around the blood vessels is small compared with the movement of the blood vessels.

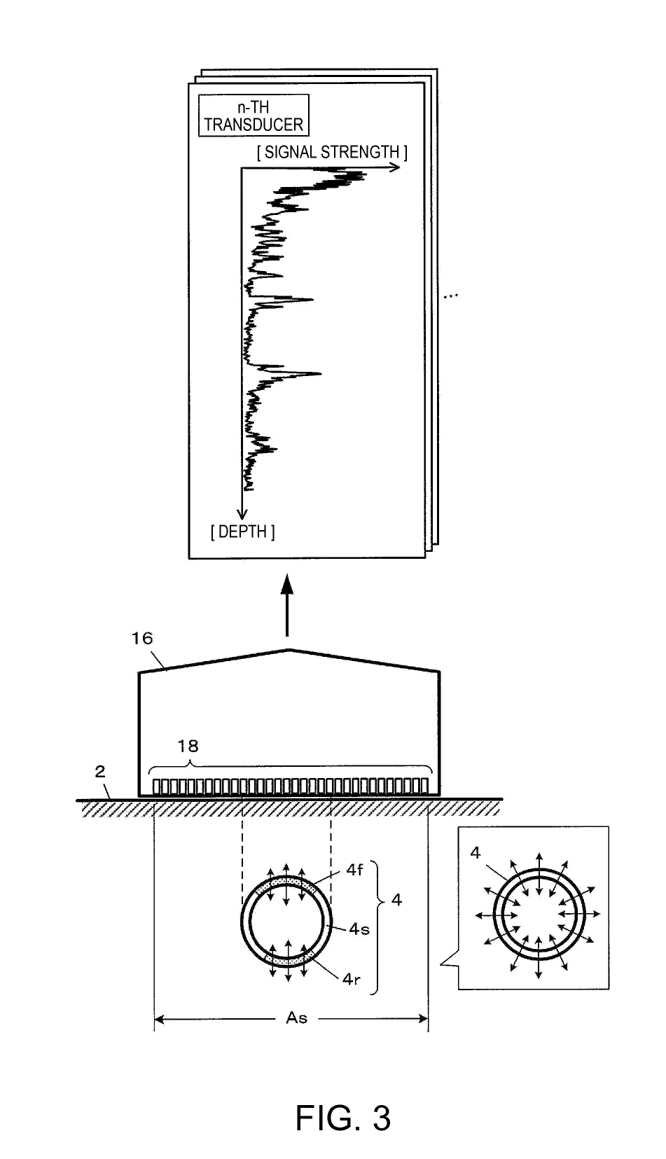

FIG. 3 is a diagram schematically showing a state where the ultrasonic probe 16 is in contact with the body surface of the subject 2 in order to perform ultrasonic measurement, and is a diagram showing the cross-section of the blood vessel 4 in a short-axis direction.

A plurality of ultrasonic transducers 18 are built into the ultrasonic probe 16. In the example shown in FIG. 3, one ultrasonic beam is emitted from each ultrasonic transducer 18 toward the bottom from the top in the diagram. The range covered by the ultrasonic transducer 18 is a probe scanning range As. The ultrasonic transducers 18 may be provided in a plurality of columns in a depth direction toward the diagram, that is, may be provided in a planar shape. Alternatively, the ultrasonic transducers 18 may be provided only in a horizontal direction with only one column in the depth direction toward the diagram.

The blood vessel 4 repeats approximately isotropic expansion/contraction due to the beating (expansion/contraction) of the heart. Therefore, a stronger reflected wave can be received as the area of the surface perpendicular to the direction of the ultrasonic beam becomes larger. However, it becomes more difficult to receive the reflected wave as the direction of the reflected wave becomes parallel to the beam direction. For this reason, in the ultrasonic measurement, the reflected wave from a front wall 4f and a rear wall 4r of the blood vessel 4 is detected strongly, but the reflected wave from a lateral wall 4s is weak. In other words, if there is the blood vessel 4 in the probe scanning range As, a strong reflected wave relevant to the front and rear walls appears in the reflected wave signal at the position of the ultrasonic transducer 18 located immediately above the blood vessel 4.

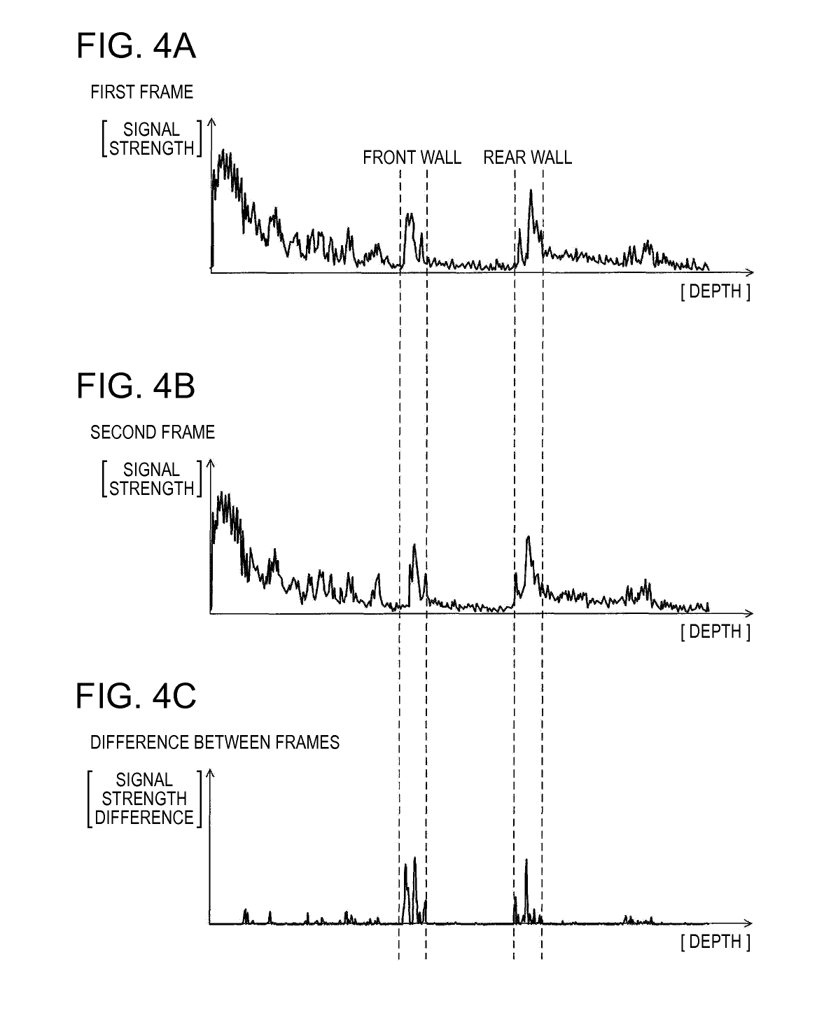

FIGS. 4A to 4C are diagrams showing an example of the received signal of the reflected wave at the position of the ultrasonic transducer 18 located immediately above the blood vessel. FIG. 4A is a “depth-signal strength graph” showing a measurement result in the first frame of the measurement period, and FIG. 4B is a “depth-signal strength graph” showing a measurement result in the second frame of the measurement period. FIG. 4C is a “graph of the signal strength difference between frames” showing a difference in the “depth-signal strength graph” between the first and second frames.

As described above, if there is the blood vessel 4, a strong reflected wave relevant to the front and rear walls is detected. Also in FIGS. 4A and 4B, peaks of two strong reflected waves that can be clearly identified appear at positions deeper than the group of reflected waves near the body surface. By calculating the signal strength difference between the first and second frames for each depth, the graph shown in FIG. 4C is obtained. Therefore, the movements of the front and rear walls of the blood vessel become clear between frames.

As is apparent from FIG. 4C, a slight signal strength difference occurs because body tissues other than the blood vessel also slightly move due to the influence of pulsation or the like. However, a large value as the value for the blood vessel (specifically, front and rear walls of the blood vessel) is not detected. Even more, such a peak is not seen in the signal strength difference graph of the reflected wave signal in the ultrasonic transducer 18 that is not located immediately above the blood vessel. That is, it can be said that the movement of the blood vessel due to pulsation appears in a change in the signal strength between frames having a time difference therebetween.

In the present embodiment, even if a change in the signal strength appropriate to the movement of the blood vessel is measured, it is not determined immediately that the ultrasonic transducer 18 is located immediately above the blood vessel, and the determination is made by statistically processing the change in the signal strength.

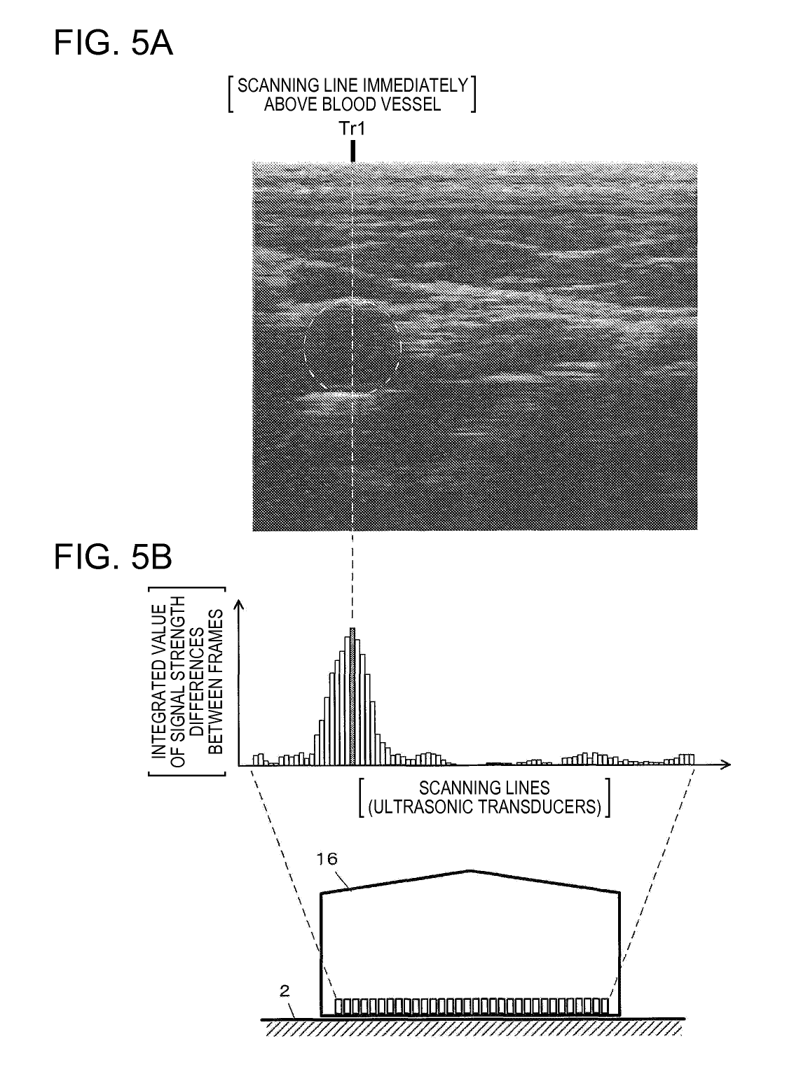

FIGS. 5A and 5B are diagrams for explaining the statistical processing on the change in the signal strength between two consecutive frames. FIG. 5A is an image obtained by converting the signal strength of the reflected wave in each ultrasonic transducer 18 into a brightness, that is, a B mode image. FIG. 5B is a histogram obtained by calculating the signal strength change in each ultrasonic transducer between two consecutive frames multiple times and integrating the signal strength changes. The point to note herein is that the horizontal axis in FIG. 4C is a depth direction and the graph is based on the reception result of one ultrasonic transducer, while the horizontal axis in FIG. 5B indicates the arrangement order of ultrasonic transducers (that is, a scanning direction and a direction along the body surface).

This will be specifically described. The histogram in FIG. 5B can be obtained by repeating calculation of the sum of the signal strength differences at all depths for each ultrasonic transducer whenever ultrasonic measurement for two consecutive frames is performed and by integrating the sums of the signal strength differences for a predetermined time (for example, at least one to several beats in a cardiac cycle: about several seconds). In other words, the histogram in FIG. 5B is a result of statistical processing in which temporal changes of the signal in the depth direction at the same position on the body surface are integrated (summed).

For the sum of the signal strength differences obtained from the ultrasonic measurement for two consecutive frames, the sum for ultrasonic transducers located on the blood vessel is a larger value than the sum for ultrasonic transducers that are not located on the blood vessel. In addition, the larger the number of ultrasonic transducers 18 located immediately above the blood vessel center, the larger the value. Needless to say, this also appears in the value on the vertical axis of the histogram obtained by integrating the sum of the signal strength differences, that is, in the integrated signal strength difference.

Accordingly, the ultrasonic transducer 18 for which the value on the vertical axis of the histogram satisfies predetermined height change conditions can be determined to be an “ultrasonic transducer located immediately above the blood vessel”. More specifically, the ultrasonic transducer 18 corresponding to the peak of the value on the vertical axis of the histogram is determined to be an “ultrasonic transducer located immediately above the blood vessel”, that is, a “scanning line immediately above the blood vessel”. In the example shown in FIGS. 5A and 5B, an ultrasonic transducer Tr1 corresponds to this.

Next, a step of detecting a vessel wall depth position candidate will be described.

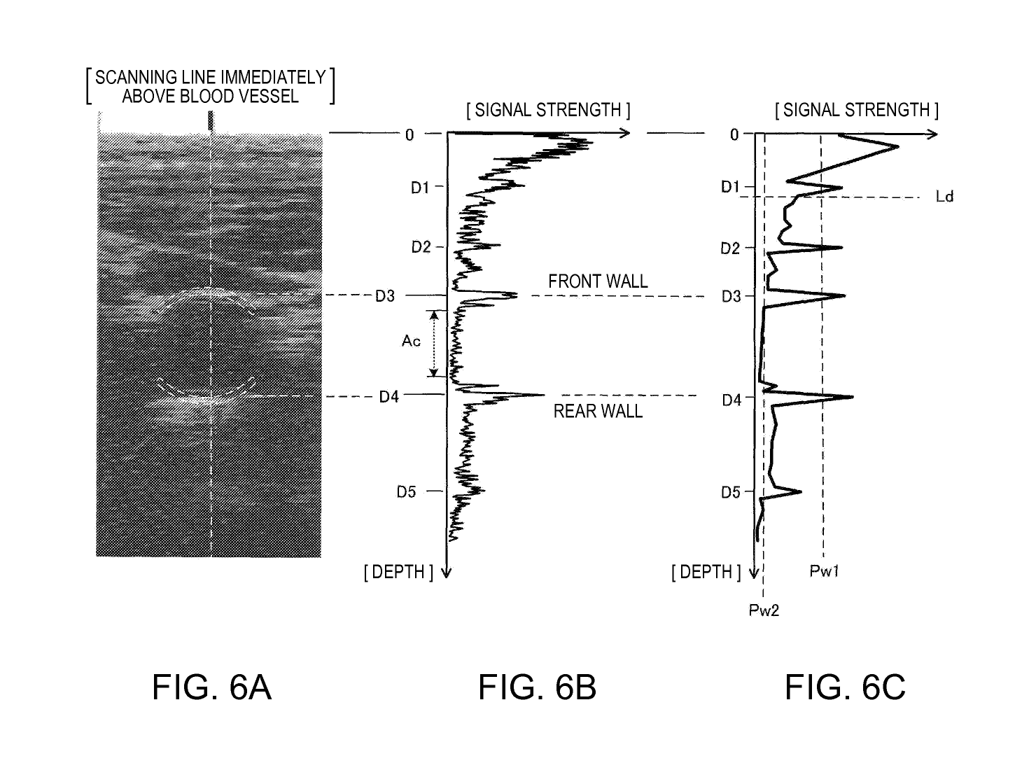

FIGS. 6A to 6C are diagrams for explaining the principle of the detection of a vessel wall depth position candidate. FIG. 6A is a B-mode image of a blood vessel part, FIG. 6B is a signal strength graph of the received signal of the reflected wave in the scanning lines immediately above the blood vessel, and FIG. 6C is a graph obtained by smoothing a change in the signal strength more clearly.

First, peaks, in which signal strengths equal to or higher than a predetermined vessel wall equivalent signal level Pw1 are obtained, are extracted. In this case, a strong reflected wave equal to or higher than the vessel wall equivalent signal level Pw1 is obtained from the front and rear walls of the blood vessel, but a strong reflected wave may also be similarly obtained from the surrounding tissues. For this reason, a plurality of peaks D1 to D5 may appear in the signal strength graph. Therefore, the peaks are narrowed down based on the likelihood of the vessel wall.

In the narrowing down, first, a peak of a shallower position than the minimum reference depth Ld is excluded from the plurality of peaks D1 to D5. The minimum reference depth Ld is the limit of shallowness at which a blood vessel having an appropriate size as a measurement target can be present, and a value larger than at least the dermis is set as the minimum reference depth Ld. In the example shown in FIGS. 6A to 6C, the peak D1 is excluded from the vessel wall depth position candidates since the depth of the peak D1 is less than the minimum reference depth Ld.

Then, the peaks are narrowed down based on the finding that the signal strength of the reflected wave of the intravascular lumen is very low compared with the surrounding tissues. That is, the peaks of the signal strength regarded as the vessel wall depth position candidates are determined as a pair of front and rear walls, and are temporarily combined. Then, the signal strengths between the respective combinations are statistically processed to calculate an average value or a median. Then, a combination satisfying the vascular front and rear walls pair equivalent conditions of “combination in which the statistical processing value is less than a predetermined intravascular lumen equivalent signal level Pw2” and “combination in which another peak is not present between the combined peaks” is extracted, and this is set as a “front and rear walls pair”.

For example, in the example of FIG. 6C, a combination in which the peak D4 is regarded as the front wall and the peak D5 is regarded as the rear wall is excluded since the statistical processing value of the signal strength between the two peaks exceeds the intravascular lumen equivalent signal level Pw2. In addition, a combination in which the peak D3 is regarded as the front wall and the peak D5 is regarded as the rear wall and a combination in which the peak D2 is regarded as the front wall and the peak D4 is regarded as the rear wall are also excluded since another peak is present between these peaks. On the other hand, a combination in which the peak D3 is regarded as the front wall and the peak D4 is regarded as the rear wall satisfies the conditions described above. Accordingly, this combination is regarded as a “front and rear walls pair”.

As a method of narrowing down, focusing on the finding that the vessel wall shows a larger movement than the surrounding tissues, determination may be made from the displacement in one cardiac cycle of the peak position of the signal strength difference between frames. In such a narrowing down method, however, for example, in a situation where there is almost no movement at the position of the front wall or the rear wall of the blood vessel in the positional relationship between the blood vessel 4 and the surrounding tissues, it is not possible to correctly narrow down the candidates for vascular front and rear walls pairs. However, according to the narrowing down method of the present embodiment, it is possible to reliably identify the vascular front and rear walls pair even in such a situation.

Next, an artery determination step will be described.

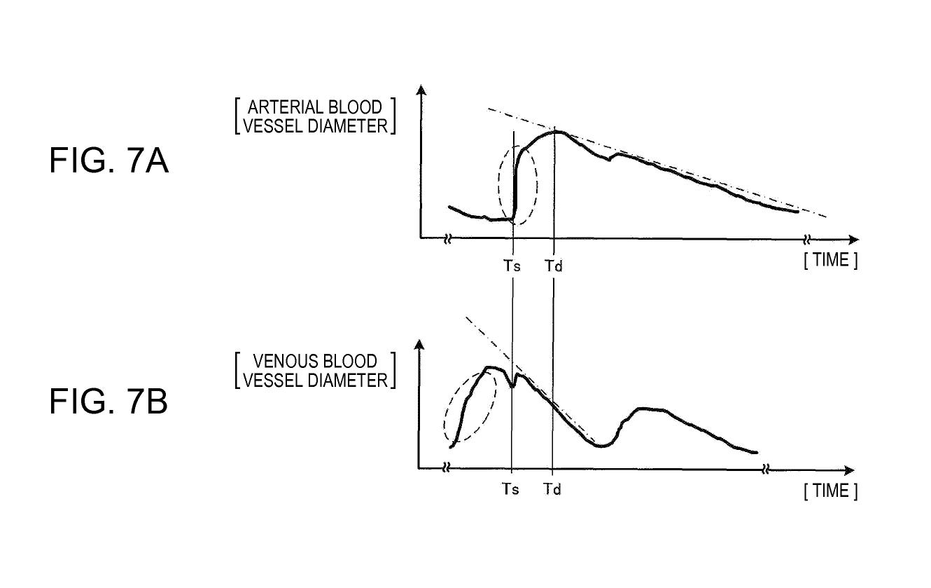

FIGS. 7A and 7B are graph showing an example of a change in the blood vessel diameter for approximately one beat of the cardiac cycle, where FIG. 7A is a graph of the arterial blood vessel diameter and FIG. 7B is a graph of the venous blood vessel diameter.

The vessel wall of the artery has a structure of high stretchability and elasticity so as to be able to withstand a pulsatile blood flow, which flows from the heart, and the blood pressure. For this reason, the blood vessel diameter increases rapidly during systole (Ts) according to the beating of the heart, and decreases slowly during diastole (Td) to return to the original thickness. Therefore, since the blood vessel diameter increases rapidly immediately after systole (Ts), the graph of the arterial blood vessel diameter rises abruptly (for example, a portion surrounded by the long dashed line in FIG. 7A). On the other hand, since the blood vessel diameter decreases slowly during diastole (Td), the graph falls gently. Thus, in the case of the artery, the degree of change in a direction in which the blood vessel diameter increases is larger than that in a direction in which the blood vessel diameter decreases, and the difference is noticeable.

On the other hand, the vessel wall (vein wall) of the vein is thinner than the vessel wall (artery wall) of the artery. Therefore, the vessel wall (vein wall) of the vein has poor elasticity. In addition, blood pressure applied to the vein wall is lower than the blood pressure applied to the artery wall. Therefore, in the case of the vein, when the degree of change in the rise (a portion surrounded by the dashed line in FIG. 7B) of the graph in a direction in which the blood vessel diameter increases is compared with the degree of change in the lowering of the graph in which the blood vessel diameter decreases, the difference as in the case of the artery does not appear.

In the present embodiment, the difference in the displacement characteristics of the vessel wall due to pulsation between the artery and the vein is identified using the displacement rate waveform of the vessel wall, and is used for artery determination.

Specifically, a temporal change in the distance between the front and rear walls, that is, the rate of change in the blood vessel diameter (hereinafter, referred to as a “diameter change rate”) is calculated by setting the position regarded as the vascular front and rear walls pair as a region of interest and calculating the displacement rate of the vessel wall from the amount of displacement per unit time using the tracking function for tracking each region of interest between different frames. Then, the artery/vein is identified from the ratio between the extreme value of a temporal change in the diameter change rate in the direction of diameter increase and the extreme value of a temporal change in the diameter change rate in the direction of diameter decrease.

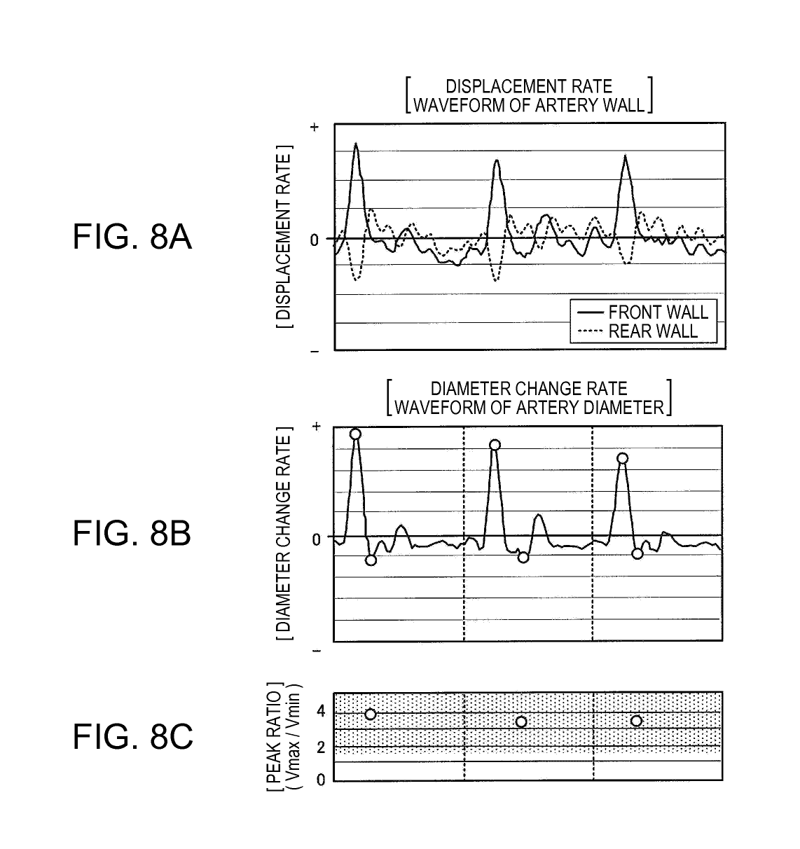

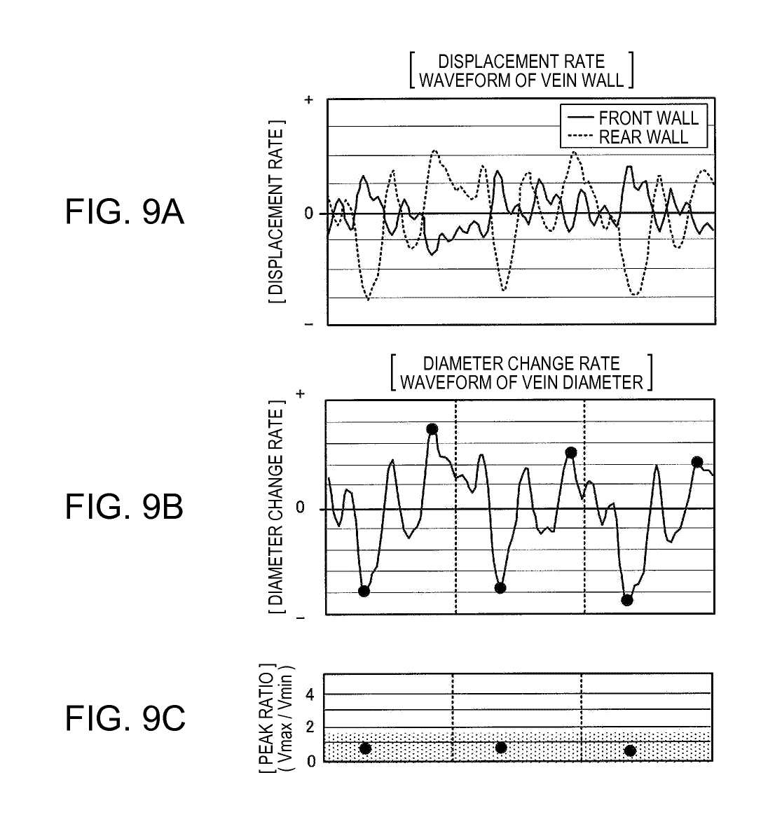

For example, FIG. 8A is a diagram showing a displacement rate waveform of the artery wall for approximately three beats of the cardiac cycle, FIG. 8B is a diagram showing a diameter change rate waveform of the artery diameter for approximately three beats of the cardiac cycle, and FIG. 8C is a diagram showing the ratio between the absolute values of extreme values (maximum and minimum values), that is, the peak ratio (maximum value/minimum value) in the diameter change rate waveform. For example, FIG. 9A is a diagram showing a displacement rate waveform of the vein wall for approximately three beats of the cardiac cycle, FIG. 9B is a diagram showing a diameter change rate waveform of the vein diameter for approximately three beats of the cardiac cycle, and FIG. 9C is a diagram showing the ratio between the absolute values of extreme values, that is, the peak ratio in the diameter change rate waveform.

The difference between the displacement characteristics of the artery wall, in which the difference between the degree of change in a direction in which the blood vessel diameter increases and the degree of change in a direction in which the blood vessel diameter decreases is noticeable, and the displacement characteristics of the vein wall, in which the difference between the degree of change in a direction in which the blood vessel diameter increases and the degree of change in a direction in which the blood vessel diameter decreases is smaller than that in the case of the artery wall, is expressed as a difference in the peak ratio, as shown in FIGS. 8C and 9C.

More specifically, the peak ratio based on the diameter change rate waveform of the diameter of the artery is relatively high, and the peak ratio based on the diameter change rate waveform of the diameter of the vein is relatively low. The boundary is generally in the range of “1.4” to “1.6”. In the present embodiment, artery/vein identification is performed by using the intermediate value “1.5” as a threshold value of the conditions that the peak ratio when the blood vessel is an artery can take. The threshold value, needless to say, can be appropriately set depending on the age range, race, sex, medical history, or the like of the subject.

Description of Functional Configuration

Next, the functional configuration for realizing the present embodiment will be described.

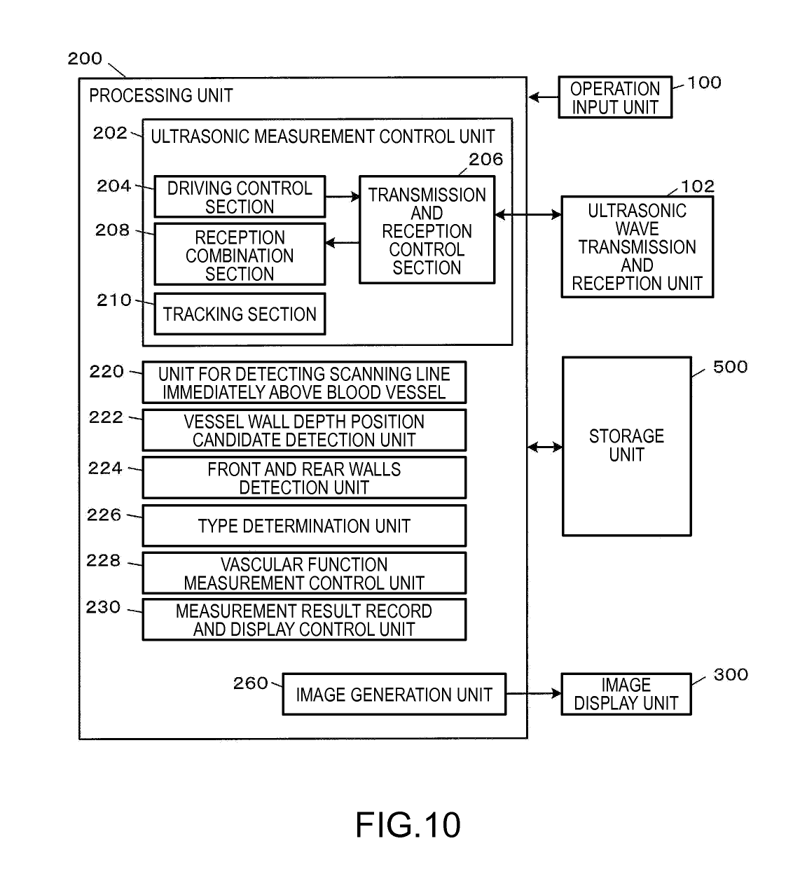

FIG. 10 is a block diagram showing an example of the functional configuration of the ultrasonic measurement apparatus 10 in the present embodiment. The ultrasonic measurement apparatus 10 includes an operation input unit 100, an ultrasonic wave transmission and reception unit 102, a processing unit 200, an image display unit 300, and a storage unit 500.

The operation input unit 100 receives various kinds of operation input by the operator, and outputs an operation input signal corresponding to the operation input to the processing unit 200. The operation input unit 100 can be implemented by a button switch, a lever switch, a dial switch, a track pad, a mouse, or the like. In the example shown in FIG. 1, the touch panel 12 or the keyboard 14 corresponds to the operation input unit 100.

The ultrasonic wave transmission and reception unit 102 transmits an ultrasonic wave with a pulse voltage output from the processing unit 200. Then, the ultrasonic wave transmission and reception unit 102 receives a reflected wave of the transmitted ultrasonic wave, converts the reflected wave into a reflected wave signal, and outputs the reflected wave signal to the processing unit 200. The ultrasonic probe 16 shown in FIG. 1 corresponds to the ultrasonic wave transmission and reception unit 102.

The processing unit 200 is realized by a microprocessor, such as a CPU or a GPU, or an electronic component, such as an ASIC or an IC memory, for example. In addition, the processing unit 200 performs control of the input and output of data to each functional unit, and calculates biological information of the subject 2 by performing various kinds of arithmetic processing based on a predetermined program or data, the operation input signal from the operation input unit 100, the reflected wave signal from the ultrasonic wave transmission and reception unit 102, or the like. The processor 30 and the control board 31 shown in FIG. 1 correspond to the processing unit 200.

In the present embodiment, the processing unit 200 includes an ultrasonic measurement control unit 202, a unit for detecting a scanning line immediately above a blood vessel 220, a vessel wall depth position candidate detection unit 222, a front and rear walls detection unit 224, a type determination unit 226, a vascular function measurement control unit 228, a measurement result record and display control unit 230, and an image generation unit 260.

The ultrasonic measurement control unit 202 controls the transmission of an ultrasonic wave toward the blood vessel and the reception of a reflected wave. For example, the ultrasonic measurement control unit 202 includes a driving control section 204, a transmission and reception control section 206, a reception combination section 208, and a tracking section 210, and performs overall control of ultrasonic measurement. The ultrasonic measurement control unit 202 can be realized by known techniques.

The driving control section 204 controls the transmission timing of ultrasonic pulses from the ultrasonic probe 16, and outputs a transmission control signal to the transmission and reception control section 206.

The transmission and reception control section 206 generates a pulse voltage according to the transmission control signal from the driving control section 204, and outputs the pulse voltage to the ultrasonic wave transmission and reception unit 102. In this case, it is possible to adjust the output timing of the pulse voltage to each ultrasonic transducer by performing transmission delay processing. In addition, it is possible to perform the amplification or filtering of the reflected wave signal output from the ultrasonic wave transmission and reception unit 102 and to output the result to the reception combination section 208.

The reception combination section 208 performs processing related to the so-called focus of a received signal by performing delay processing as necessary, thereby generating reflected wave data.

The tracking section 210 performs processing related to so-called “tracking” that is for tracking the position of a region of interest between frames of ultrasonic measurement based on the reflected wave data (reflected wave signal). For example, it is possible to perform processing for setting a region of interest (tracking point) in the reflected wave data (for example, a B-mode image) as a reference, processing for tracking each region of interest between different frames, and processing for calculating the displacement for each region of interest. Functions, such as so-called “echo tracking” or “phase difference tracking” that is known, are realized.

The unit for detecting a scanning line immediately above a blood vessel 220 performs arithmetic processing for detecting the scanning lines immediately above the blood vessel or controls each unit. That is, control relevant to the above-described step of detecting the scanning lines immediately above the blood vessel is performed (refer to FIGS. 5A and 5B).

The vessel wall depth position candidate detection unit 222 detects a depth position regarded as a vessel wall based on the received signal of the reflected wave in the scanning lines immediately above the blood vessel. A part of control relevant to the above-described step of detecting the vessel wall depth position candidate is performed (refer to FIG. 6B).

The front and rear walls detection unit 224 detects the front and rear walls of the blood vessel using the received signal of the reflected wave in the scanning lines immediately above the blood vessel. A part of control relevant to the above-described step of narrowing down the front and rear walls pair of the blood vessel is performed (refer to FIG. 6C).

The type determination unit 226 determines the type of the artery/vein using a temporal change in the distance between the front and rear walls. A part of control relevant to the artery determination step described above is performed (refer to FIGS. 7A to 9C).

When the type determination unit 226 determines that the blood vessel is an artery, the vascular function measurement control unit 228 performs control relevant to predetermined vascular function measurement by continuing position measurement with the front and rear walls of the blood vessel as a tracking target.

The measurement result record and display control unit 230 performs control for storing the measurement result of the vascular function in the storage unit 500 and displaying the measurement result of the vascular function on the image display unit 300.

The image generation unit 260 generates an image for displaying a measurement result or various operation screens required for ultrasonic measurement or biological information measurement, and outputs the image to the image display unit 300.

The image display unit 300 displays image data input from the image generation unit 260. The touch panel 12 shown in FIG. 1 corresponds to the image display unit 300.

The storage unit 500 is realized by a storage medium, such as an IC memory, a hard disk, or an optical disc, and stores various programs or various kinds of data, such as data in the operation process of the processing unit 200. In FIG. 1, the storage medium 33 mounted in the control board 31 of the processor 30 corresponds to the storage unit 500. In addition, the connection between the processing unit 200 and the storage unit 500 is not limited to a connection using an internal bus circuit in the apparatus, and may be realized by using a communication line, such as a local area network (LAN) or the Internet. In this case, the storage unit 500 may be realized by using a separate external storage device from the ultrasonic measurement apparatus 10.

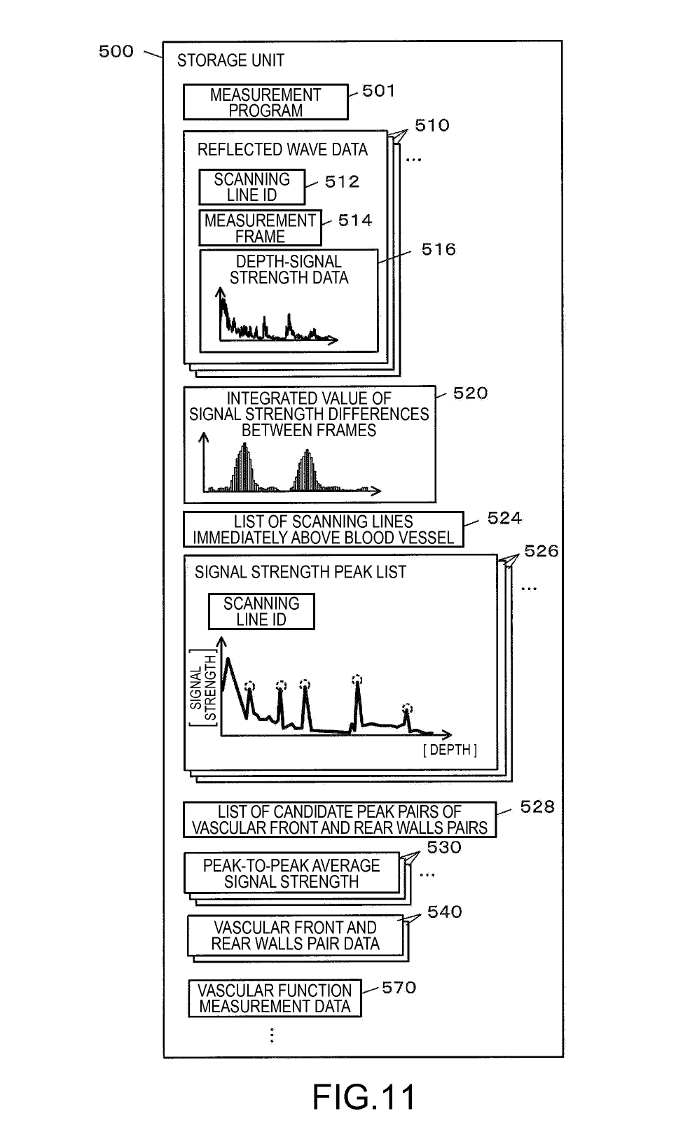

In addition, as shown in FIG. 11, the storage unit 500 stores a measurement program 501, reflected wave data 510, an integrated value of signal strength differences between frames 520, and a list of scanning lines immediately above a blood vessel 524, a signal strength peak list 526, a list of candidate peak pairs of vascular front and rear walls pairs 528, a peak-to-peak average signal strength 530, vascular front and rear walls pair data 540, and vascular function measurement data 570. Needless to say, frame identification information, various flags, counter values for time checking, and the like other than those described above can also be appropriately stored.

The processing unit 200 realizes the functions of the ultrasonic measurement control unit 202, the unit for detecting a scanning line immediately above a blood vessel 220, the vessel wall depth position candidate detection unit 222, the front and rear walls detection unit 224, the type determination unit 226, the vascular function measurement control unit 228, the measurement result record and display control unit 230, the image generation unit 260, and the like by reading and executing the measurement program 501. In addition, when realizing these functional units with hardware, such as an electronic circuit, a part of the program for realizing the function can be omitted.

The reflected wave data 510 is reflected wave data obtained by ultrasonic measurement, and is generated for each frame by the ultrasonic measurement control unit 202. Apiece of reflected wave data 510 includes, for example, a scanning line ID 512, a measurement frame 514, and depth-signal strength data 516. Needless to say, data other than those described above can also be appropriately stored (refer to FIGS. 3 to 4C).

The integrated value of signal strength differences between frames 520 is data of a histogram obtained by repeating calculation of the sum of the signal strength differences at all depths for each ultrasonic transducer whenever ultrasonic measurement for two consecutive frames of the reflected wave data 510 is performed and by integrating the sum of the signal strength differences for a predetermined time (refer to FIG. 5B).

The list of scanning lines immediately above a blood vessel 524 is a list of scanning line IDs or ultrasonic transducer IDs determined to be scanning lines immediately above the blood vessel.

The signal strength peak list 526 is generated for each scanning line immediately above the blood vessel, and is a list of peaks of the depth position candidates regarded as vessel walls that have been read from the depth-signal strength data in the scanning line, that is, a list of peaks of the signal strengths (refer to the peaks D1 to D5 in FIG. 6B).

The list of candidate peak pairs of vascular front and rear walls pairs 528 is data generated in the process of narrowing down the pairs of front and rear walls as a vascular front and rear walls pair from the depth position candidates regarded as vessel walls, and is a list of combinations of a peak regarded as a front wall and a peak regarded as a rear wall.

The peak-to-peak average signal strength 530 is generated for each pair of peaks registered in the list of candidate peak pairs of vascular front and rear walls pairs 528, and the statistical value of the signal strength between the peaks of the pair (refer to “between peaks Ac” in FIG. 6B) is stored.

The vascular front and rear walls pair data 540 is prepared for each vascular front and rear walls pair, and stores the depth position of each pair of front and rear walls or various kinds of information required for the identification of blood vessels of the vascular front and rear walls pair. In the present embodiment, since it is assumed that the ultrasonic probe 16 is in contact with a carotid artery part, the vascular front and rear walls pair data 540 of two parts of the artery and the vein are shown in the example of FIG. 11. In practice, however, the vascular front and rear walls pair data 540 are stored as many as the number of blood vessels included in the scanning range of the ultrasonic probe 16.

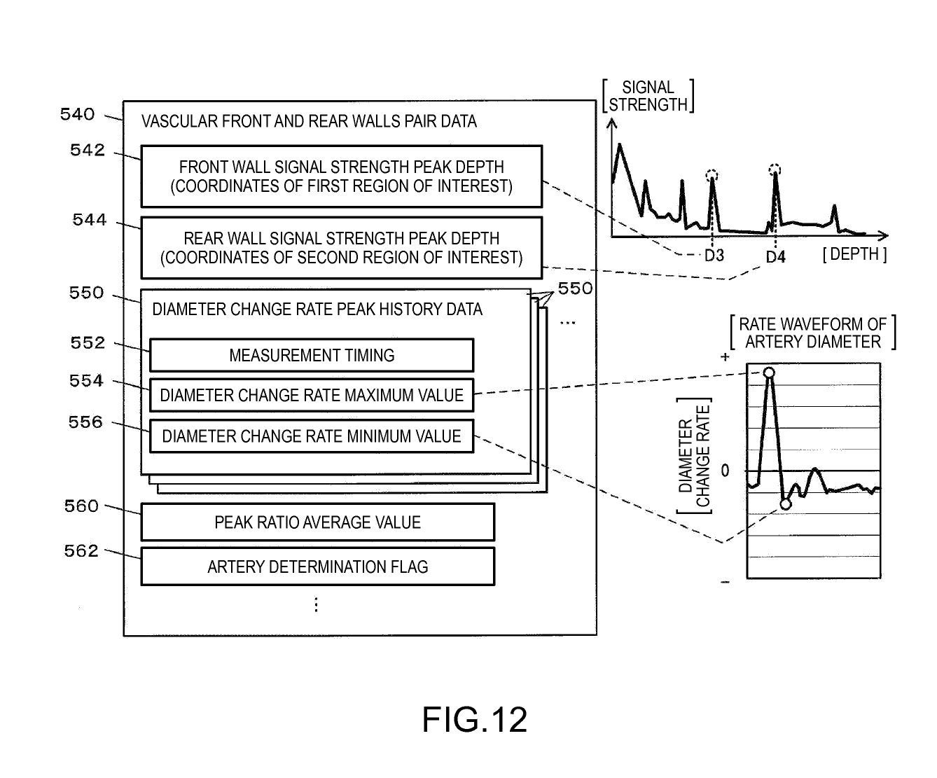

For example, as shown in FIG. 12, apiece of vascular front and rear walls pair data 540 includes a front wall signal strength peak depth 542, a rear wall signal strength peak depth 544, diameter change rate peak history data 550, a peak ratio average value 560, and an artery determination flag 562.

The front wall signal strength peak depth 542 and the rear wall signal strength peak depth 544 are depth positions of the peaks of the signal strengths regarded as front and rear walls, and correspond to the coordinates of a first region of interest and the coordinates of a second region of interest in the tracking control for artery determination, respectively.

In the diameter change rate peak history data 550, an extreme value of the diameter change rate waveform in one beat of the cardiac cycle of the blood vessel having the vascular front and rear walls pair is stored. In a piece of diameter change rate peak history data 550, for example, measurement timing 552, a diameter change rate maximum value 554 of the blood vessel diameter, and a diameter change rate minimum value 556 of the blood vessel diameter are stored.

In the peak ratio average value 560, the average value of the peak ratio (diameter change rate maximum value 554/diameter change rate minimum value 556) calculated for each piece of the diameter change rate peak history data 550 is further stored.

The artery determination flag 562 is a flag in which “1” is stored when determination as an artery is made.

Description of the Flow of the Process

Next, the operation of the ultrasonic measurement apparatus 10 in each step from the detection of the scanning lines immediately above the blood vessel to the artery determination processing will be described (refer to FIG. 2).

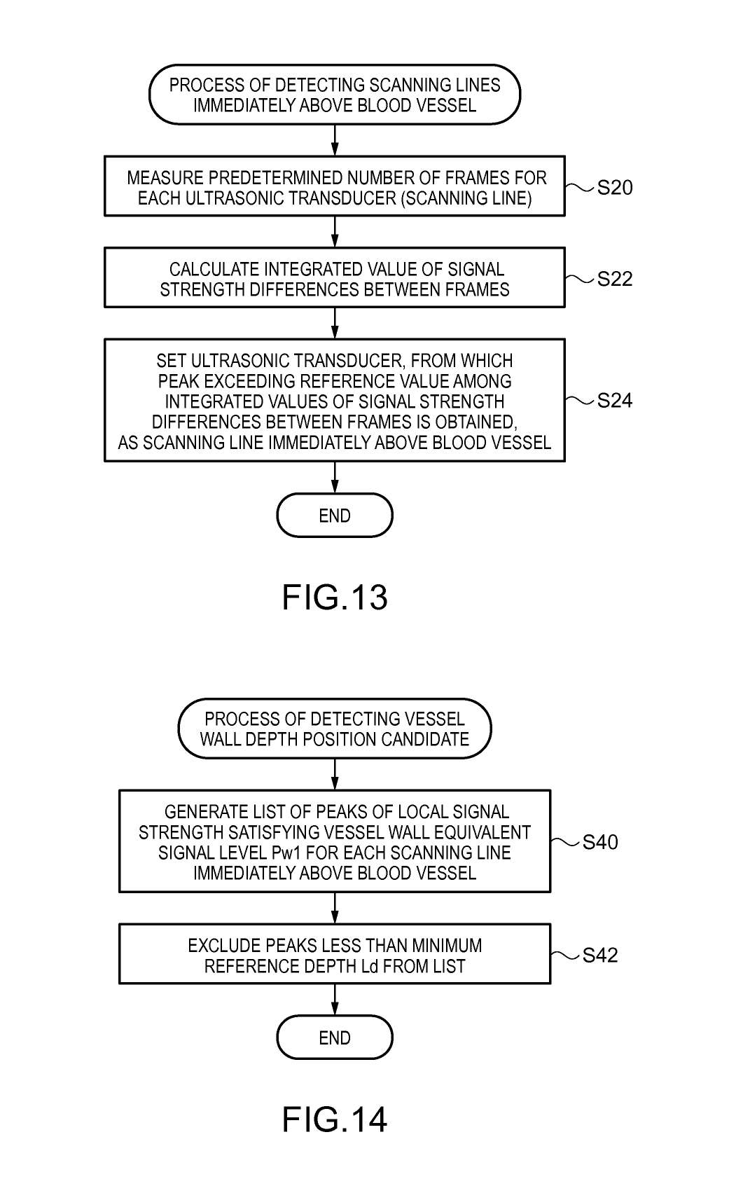

FIG. 13 is a flowchart for explaining the flow of the process of detecting the scanning lines immediately above the blood vessel in the ultrasonic measurement apparatus 10 according to the present embodiment.

In this process, first, the processing unit 200 transmits ultrasonic beams of a predetermined number of frames to each ultrasonic transducer (scanning line) and receives the reflected waves (step S20). Then, the reflected wave data 510 (refer to FIG. 11) is stored in the storage unit 500.

Then, the integrated value of signal strength differences between frames 520 (refer to FIGS. 5A and 5B) is calculated from the reflected wave data 510 (step S22). Then, an ultrasonic transducer from which a peak exceeding a predetermined reference value is obtained is determined to be the scanning line immediately above the blood vessel, and the scanning line ID corresponding to the ultrasonic transducer is registered in the list of scanning lines immediately above a blood vessel 524 (refer to FIG. 11) (step S24). Then, the process of detecting the scanning lines immediately above the blood vessel is ended.

FIG. 14 is a flowchart for explaining the flow of the process of detecting the vessel wall depth position candidates in the ultrasonic measurement apparatus 10 according to the present embodiment.

In this process, the processing unit 200 extracts a local peak, in which the signal strength satisfies the predetermined vessel wall equivalent signal level Pw1 (refer to FIGS. 5A and 5B), from the reflected wave data 510 of the scanning line for each scanning line immediately above the blood vessel that is registered in the list of scanning lines immediately above a blood vessel 524, thereby generating the signal strength peak list 526 for each scanning line immediately above the blood vessel (step S40). Then, peaks of the signal strength less than the minimum reference depth Ld are excluded from the list (step S42), and the process of detecting the vessel wall depth position candidates is ended.

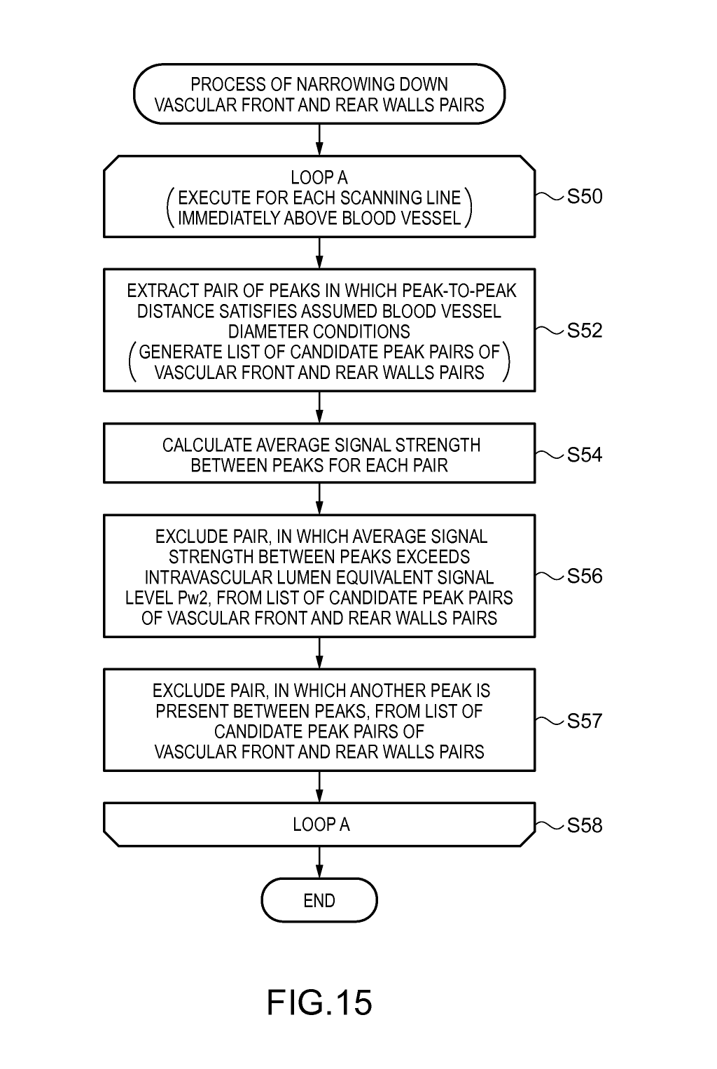

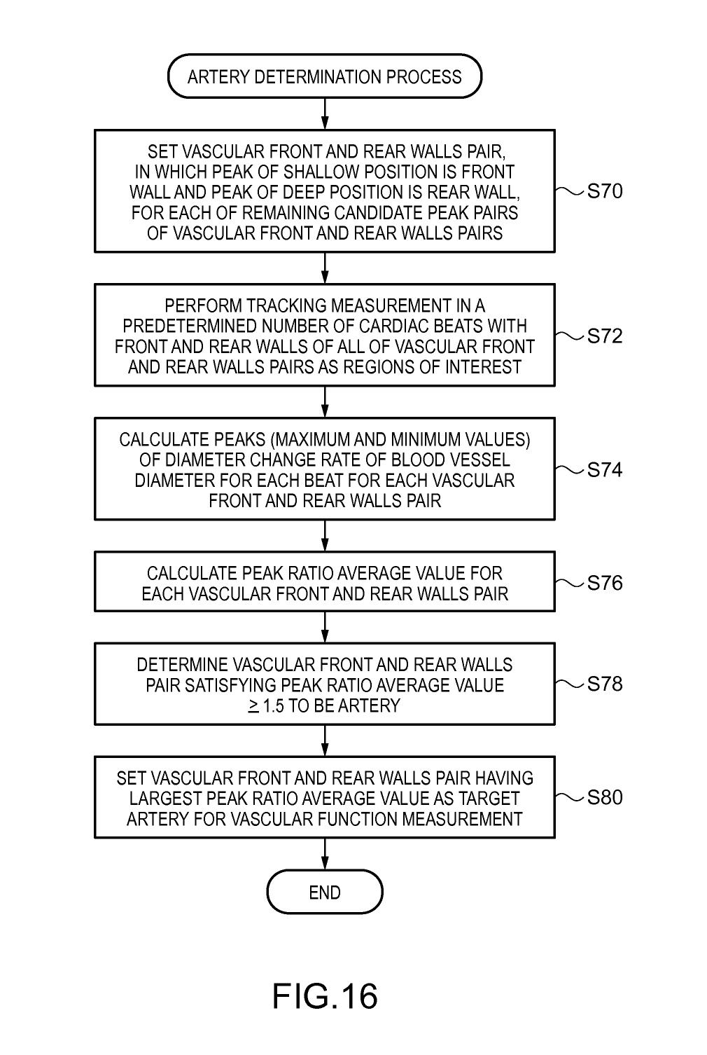

FIG. 15 is a flowchart for explaining the flow of the process of narrowing down the vascular front and rear walls pairs in the ultrasonic measurement apparatus 10 according to the present embodiment.

In this process, the processing unit 200 executes a loop A for each scanning line immediately above the blood vessel that is registered in the list of scanning lines immediately above a blood vessel 524 (steps S50 to S58).

In the loop A, first, the processing unit 200 generates a pair from the registered peaks with reference to the signal strength peak list 526 corresponding to the scanning lines immediately above the blood vessel to be processed, and extracts a pair in which a peak-to-peak distance satisfies predetermined assumed blood vessel diameter conditions, thereby generating the list of candidate peak pairs of vascular front and rear walls pairs 528 (step S52). The assumed blood vessel diameter conditions referred to herein are conditions defining a rough range of the blood vessel diameter suitable for the measurement, and it is assumed that the assumed blood vessel diameter conditions are set in advance by tests or the like.

Then, an average signal strength between peaks is calculated for each pair of peaks registered in the list of candidate peak pairs of vascular front and rear walls pairs 528 (step S54), and a pair in which the average signal strength between peaks exceeds the intravascular lumen equivalent signal level Pw2 (refer to FIGS. 5A and 5B) is excluded from the list of candidate peak pairs of vascular front and rear walls pairs 528 (step S56).

In addition, among the peaks registered in the list of candidate peak pairs of vascular front and rear walls pairs 528, a pair in which another peak is present between peaks is excluded from the list (step S56), and the loop A is ended (step S58). A pair of peaks remaining in the list of candidate peak pairs of vascular front and rear walls pairs 528 in this stage is front and rear walls of the blood vessel in the scanning lines immediately above the blood vessel to be processed.

FIG. 15 is a flowchart for explaining the flow of the artery determination process in the ultrasonic measurement apparatus 10 according to the present embodiment.

In this process, first, for each pair of peaks remaining in the list of candidate peak pairs of vascular front and rear walls pairs 528, the processing unit 200 generates the vascular front and rear walls pair data 540 (refer to FIG. 12) by regarding the peak of the relatively shallow position (position where the depth from the body surface is small) of the pair as a front wall and the peak of the relatively deep position as a rear wall (step S70).

Then, the processing unit 200 sets the front wall signal strength peak depth 542 and the rear wall signal strength peak depth 544 of all pieces of the vascular front and rear walls pair data 540 as a region of interest, and tracks the displacement of each region of interest in a predetermined number of cardiac beats (step S72). It is also possible to use the reflected wave data 510 that is already stored. Then, for each vascular front and rear walls pair, the peak of the diameter change rate of the blood vessel diameter is calculated for each beat of the cardiac cycle, thereby generating the diameter change rate peak history data 550 (step S74).

Then, the processing unit 200 calculates the peak ratio average value 560 for each vascular front and rear walls pair (step S76). Then, the processing unit 200 determines a vascular front and rear walls pair, which has a peak ratio equal to or greater than a predetermined threshold value (in the present embodiment, 1.5), to be an artery and sets the artery determination flag 562 to “1”, and determines a vascular front and rear walls pair having a peak ratio less than the predetermined threshold value and sets the artery determination flag 562 to “0” (step S78). Then, the processing unit 200 sets a vascular front and rear walls pair having the largest peak ratio average value 560 as a target artery for vascular function measurement (step S80), and the artery determination process is ended.

As described above, according to the present embodiment, it is possible to find an artery automatically from the tissues in the body in the scanning range As (refer to FIG. 3) of the ultrasonic probe 16 and to perform vascular function measurement with the artery as a measurement target. Therefore, since the only thing that the operator has to do is to place the ultrasonic probe 16 at an approximate place where the carotid artery may be present, labor in the measurement work is greatly reduced. As a result, measurement errors can also be reduced significantly.

In addition, embodiments of the invention are not limited to the embodiment described above, and constituent components can be appropriately added, omitted, and changed.

For example, the displacement rate in the embodiment described above can be appropriately replaced with displacement acceleration.

The entire disclosure of Japanese Patent Application No. 2014-015167, filed on Jan. 30, 2014 is expressly incorporated by reference herein.

What is claimed is:

1. An ultrasonic measurement apparatus, comprising:
a transmission and reception control unit that controls transmission of ultrasonic waves to a blood vessel and reception of reflected waves that are reflected from the blood vessel;
a front and rear walls detection unit that detects front and rear walls of the blood vessel using received signals of the reflected waves; and
a type determination unit that determines a type of the blood vessel using a temporal change in a distance between the front and rear walls.

2. The ultrasonic measurement apparatus according to claim 1,
wherein the type determination unit determines a type of the blood vessel using a temporal change in the distance in a direction of increase and a temporal change in the distance in a direction of decrease.

3. The ultrasonic measurement apparatus according to claim 2,
wherein the type determination unit determines a type of the blood vessel using a ratio between an extreme value of the temporal change in the direction of increase and an extreme value of the temporal change in the direction of decrease.

4. The ultrasonic measurement apparatus according to claim 3,
wherein the type determination unit determines that the blood vessel is an artery using at least a value that the ratio can have when the blood vessel is an artery

5. The ultrasonic measurement apparatus according to claim 1,
wherein the front and rear walls detection unit detects front wall candidates and rear wall candidates for the blood vessel using the received signals, and selects a pair satisfying predetermined conditions, among pairs of the front wall candidates and the rear wall candidates, as the front and rear walls of the blood vessel.

6. The ultrasonic measurement apparatus according to claim 5,
wherein the front and rear walls detection unit selects the front and rear walls of the blood vessel based on the predetermined conditions including at least a condition that a signal between each of the front wall candidates and each of the rear wall candidates, among the received signals, satisfies predetermined intravascular equivalent conditions.

7. The ultrasonic measurement apparatus according to claim 1, further comprising:
a vascular function measuring unit that performs predetermined vascular function measurement by continuing position measurement with the front and rear walls of the blood vessel as tracking targets when the blood vessel is determined to be an artery by the type determination unit.

8. An ultrasonic measurement method, comprising:
controlling transmission of ultrasonic waves to a blood vessel and reception of reflected waves that are reflected from the blood vessel;
detecting front and rear walls of the blood vessel using received signals of the reflected waves; and
determining a type of the blood vessel using a temporal change in a distance between the front and rear walls.
